(19) United States
(12) Patent Application Publication
(10) Pub. No.: US 2013/0217082 A1
(43) Pub. Date: Aug. 22, 2013

(54) Algae Biofuel Carbon Dioxide Distribution System

(71) Applicant: David A. Hazlebeck, El Cajon, CA (US)

(72) Inventor: David A. Hazlebeck, El Cajon, CA (US)

(21) Appl. No.: 13/589,951

(22) Filed: Aug. 20, 2012

Related U.S. Application Data
(63) Continuation-in-part of application No. 11/549,541, filed on Oct. 13, 2006, now Pat. No. 8,262,776.
Application No. 11/549,541 is a continuation-in-part of application No. 12/817,029, filed on Jun. 16, 2010, now abandoned.

(51) Int. Cl. C12P 7/64

(52) U.S. Cl. CPC C12P 7/649
USPC 435/134; 435/292.1

(57) ABSTRACT

A closed-loop system for growing algae in a bioreactor is disclosed, for example, to produce biofuel. An aqueous bioreactor solution is formulated such that the principal source of carbon for algae growth is supplied by sodium bicarbonate. During algae growth in the aqueous solution, the concentration of sodium bicarbonate in the solution is reduced while the concentration of sodium carbonate increases. A regenerator is provided to regenerate sodium bicarbonate from the sodium carbonate. Specifically, after sufficient growth, the algae are concentrated and an algae depleted media is produced. Carbon dioxide is then introduced into the algae depleted media to regenerate sodium bicarbonate from the sodium carbonate. The regenerated sodium bicarbonate is then recycled into the bioreactor to supply carbon for further algae growth.

This application is a continuation-in-part of application Ser. No. 11/549,541, filed Oct. 13, 2006, which is currently pending. This application is also a continuation-in-part application of application Ser. No. 12/817,029, filed Jun. 16, 2010, which is currently pending. The contents of application Ser. No. 11/549,541 and application Ser. No. 12/817,029 are incorporated herein by reference.

### FIELD OF THE INVENTION

The present invention pertains generally to processes for growing algae. More particularly, the present invention pertains to the processes for growing algae in an aqueous solution of sodium bicarbonate. The present invention is particularly, but not exclusively, useful as a closed loop system and method that removes spent media from an algae growing bioreactor, regenerates sodium bicarbonate in the media and recycles the media back into the system to sustain further algae growth.

### BACKGROUND OF THE INVENTION

As worldwide petroleum deposits decrease, there is rising concern over shortages and the costs that are associated with the production of hydrocarbon products. As a result, alternatives to products that are currently processed from petroleum are being investigated. In this effort, biofuel such as biodiesel has been identified as a possible alternative to petroleum-based transportation fuels. In general, a biodiesel is a fuel comprised of mono-alkyl esters of long chain fatty acids derived from plant oils or animal fats. In industrial practice, biodiesel is created when plant oils or animal fats react with an alcohol, such as methanol.

For plant-derived biofuel, solar energy is first transformed into chemical energy through photosynthesis. The chemical energy is then refined into a usable fuel. Currently, the process involved in creating biofuel from plant oils is expensive relative to the process of extracting and refining petroleum. It is possible, however, that the cost of processing a plant-derived biofuel could be reduced by maximizing the rate of growth of the plant source and by minimizing the costs of feeds needed to support the plant growth. Because algae are known to be one of the most efficient plants for converting solar energy into cell growth, it is of particular interest as a biofuel source. However, current algae processing methods have failed to result in a cost effective algae-derived biofuel.

In overview, the biochemical process of photosynthesis provides algae with the ability to convert solar energy into chemical energy. During cell growth, this chemical energy is used to drive synthetic reactions, such as the formation of sugars or the fixation of nitrogen into amino acids for protein synthesis. Excess chemical energy is stored in the form of fats and oils as triglycerides. Thus, the creation of oil in algae only requires sunlight, carbon dioxide and the nutrients necessary for formation of triglycerides. Nevertheless, with the volume requirements for a fuel source, the costs associated with the inputs are high.

One possible source of carbon dioxide and other nutrients that support cell growth is found in flue gases from power plants or other combustion sources. Further, when present in flue gases, these nutrients are considered pollutants that must be properly disposed of. Therefore, use of nutrients from flue gases to support cell growth will abate pollution. However, the introduction of carbon dioxide directly into the media of an algae growth system can be complicated, and in some cases, prohibitively expensive. For example, some designs call for a large, piping system to distribute carbon dioxide throughout a large algae biofuel production facility. In addition, losses from off-gassing of carbon dioxide can be excessive.

In light of the above, it is an object of the present invention to provide a system and method for producing algae-derived biofuel which reduces input costs. Another object of the present invention is to provide a system and method for producing algae-derived biofuel that causes pollution abatement. Still another object of the present invention is to provide a system for supplying nutrients to algae cells in the form of pollutants scrubbed from flue gases. Another object of the present invention is to provide a system for recycling the effluent from a medium for growing algae as a scrubber solution. Another object of the present invention is to provide a system for producing algae-derived biofuel that defines a flow path for continuous movement of the algae, its processed derivatives, and the medium fostering its growth. Still another object of the present invention is to provide a system which allows for the effective use of carbon dioxide that is introduced at a single location and reduces/eliminates carbon dioxide losses due to off-gassing. Yet another object of the present invention is to provide an algae biofuel carbon dioxide distribution system and method that are simple to implement, easy to use, and comparatively cost effective.

### SUMMARY OF THE INVENTION

In accordance with the present invention, a closed-loop system for growing algae includes a bioreactor for growing algae cells in an aqueous solution. In particular, the algae that are grown in the system can be used to produce biofuel. For the system, the solution is formulated such that the principal source of carbon for algae growth is supplied by sodium bicarbonate. For example, in some implementations, at least 50 percent of the carbon supplied for algae growth is supplied by sodium bicarbonate. Preferably, over 90 percent of the carbon supplied for algae growth is supplied by sodium bicarbonate.

During algae growth in the aqueous solution, the concentration of sodium bicarbonate in the solution is reduced by the algae growth, while the concentration of sodium carbonate increases. To benefit from this conversion, a regenerator is provided in the system to regenerate sodium bicarbonate from the sodium carbonate. In more detail, a separator is included in the system for concentrating cultivated algae. This results in concentrated algae, and an algae depleted media. Once it is separated from the algae, the algae depleted media can be treated to regenerate sodium bicarbonate from the sodium carbonate. More specifically, carbon dioxide can be introduced into the algae depleted media to regenerate sodium bicarbonate from the sodium carbonate. The regenerated sodium bicarbonate can then be directed back into the bioreactor to supply carbon for further algae growth.

In one arrangement, a scrubber can be used to introduce carbon dioxide into the algae depleted media. For this arrangement, a scrubber solution made up of the algae depleted media can be used to scrub a power plant effluent containing carbon dioxide. In other embodiments, some or all of the carbon dioxide that is introduced into the algae depleted media can be either in the form of liquid carbon dioxide, carbon dioxide from an ethanol plant, carbon dioxide from a gasification source or carbon dioxide from a combustion source. In addition to carbon dioxide, heterotrophic sources of carbon such as algae debris, glycerin or cellulosic sugar can be introduced into the algae depleted media in the regenerator.

In a particular embodiment of the invention, a system is provided for producing high oil content biofuel from algae fed with pollutants. In this manner, the system serves to produce an environmentally-friendly fuel while abating pollution. Structurally, the system includes a scrubber having a chamber for receiving a pollutant-contaminated fluid stream and a scrubber solution. Typically, the fluid stream comprises flue gas from a combustion source, such as a power plant, which is polluted with carbon dioxide, sulfur oxides, and/or nitrogen oxides. Further, the scrubber solution is typically a caustic or sodium bicarbonate.

For purposes of the present invention, the system also includes a bioreactor for growing algae cells with high oil content. Structurally, the bioreactor includes at least one chemostat and a plug flow reactor. More particularly, the chemostat is a continuously-stirred flow reactor that has an input port, a conduit, and an output port. Preferably, the conduit is formed by an endless, open raceway that receives and holds a medium, and a paddlewheel spanning the conduit is provided to circulate the medium through the conduit. For purposes of the present invention, the plug flow reactor is positioned relative to the chemostat to receive overflow medium containing algae cells from the chemostat. Specifically, the plug flow reactor includes an input port that receives the overflow medium from the output port of the chemostat. Further, the plug flow reactor is in the farm of an open raceway that includes a conduit for continuously moving the medium downstream under the influence of gravity.

In addition to the scrubber and bioreactor, the system includes an algae separator. Specifically, the algae separator is positioned in fluid communication with the plug flow reactor to remove the algae cells from the plug flow reactor's conduit. Downstream of the algae separator, the system includes a channel for recycling an effluence from the plug flow reactor to the scrubber for reuse as the scrubber solution. Further, the system includes an apparatus for lysing algae cells to unbind oil from the algae cells. For the present invention, the lysing apparatus is positioned to receive algae cells from the algae separator. Downstream of the lysing apparatus, the system includes an oil separator that receives the lysed cells and withdraws the oil from remaining cell matter. The oil separator has an outlet for the remaining cell matter which is in fluid communication with the chemostat. Further, the system may include a hydrolyzing device that is interconnected between the oil separator and the chemostat. In addition to the cell matter outlet, the oil separator includes an outlet for the oil in fluid communication with a biofuel reactor. In a known process, the biofuel reactor causes an alcohol to react with the oil to synthesize biofuel and, as a byproduct, glycerin. Structurally, the biofuel reactor includes a glycerin exit that is in fluid communication with the plug flow reactor.

In operation, the flue gas from the power plant is flowed through the chamber of the scrubber. At the same time, the scrubber solution is sprayed into the scrubber chamber to trap the pollutants in the flue gas. The scrubber solution with the entrapped pollutants is then delivered to the chemostat through its input port. Also, a nutrient mix may be fed into the chemostat through the input port to form, along with the scrubber solution, a medium for growing algae cells. As the paddlewheel circulates the medium through the conduit of the chemostat, the algae cells grow using solar energy and converting the pollutants and other nutrients to cell matter. Preferably, a continuous flow of the medium washes the algae cells and constantly removes them from the chemostat as overflow.

After the overflow medium is removed from the chemostat, it is received in the plug flow reactor and is treated in order to trigger the production of oil in the form of triglycerides in the algae cells. After passing along the conduit of the plug flow reactor, the effluent, including algae cells, passes through the algae separator which removes the algae cells from the effluent. Thereafter, the effluent is recycled through a channel back to the scrubber for reuse as the scrubber solution. At the same time, the algae cells are delivered to the cell lysis apparatus. Then, the cell lysis apparatus lyses the cells to unbind the oil from the remaining cell matter. This unbound cell material is received by the oil separator from the cell lysis apparatus. Next, the oil separator withdraws the oil from the remaining cell matter and effectively forms two streams of material. The stream of remaining cell matter is transferred to the hydrolysis apparatus where the cell matter is broken into small units which are more easily absorbed by algae cells during cell growth. Thereafter, the hydrolyzed cell matter is delivered to the chemostat to serve as a source of nutrition for the algae cells growing therein. At the same time, the stream of oil is transmitted from the oil separator to the biofuel reactor. In the biofuel reactor, the oil is reacted with an alcohol to form biofuel and a glycerin byproduct. The glycerin byproduct is fed back into the plug flow reactor to serve as a source of carbon for the algae cells therein during the production of intracellular oil.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of a system for producing biofuel from pollutant-fed algae in accordance with the present invention; and

FIG. 2 is a schematic view of a closed loop system for growing algae that regenerates sodium bicarbonate in spent bioreactor media and recycles the regenerated media back into the system to sustain further algae growth.

### DESCRIPTION OF THE PREFERRED EMBODIMENTS

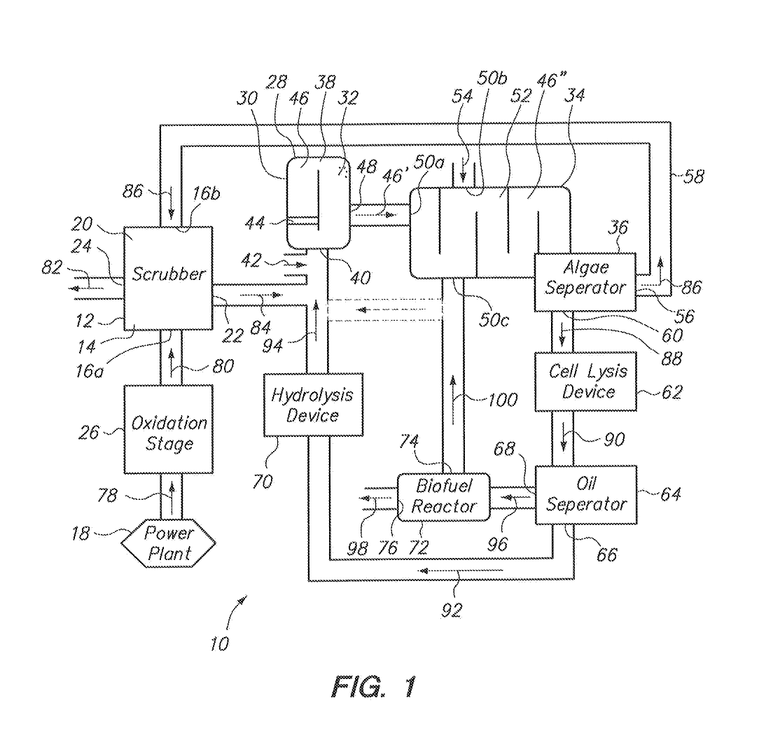

Referring initially to FIG. 1, a system for producing biofuel from pollutant-fed algae in accordance with the present invention is shown and generally designated 10. As shown, the system 10 includes a scrubber 12 for scrubbing a pollutant-contaminated fluid stream. Specifically, the scrubber 12 includes a chamber 14 and an input port 16a for receiving flue gas from a combustion source such as a power plant 18 and a scrubber solution 20. Typically, the flue gas includes pollutants such as carbon dioxide, sulfur oxides, and/or nitrogen oxides. Also, the scrubber solution 20 typically comprises sodium hydroxide or sodium bicarbonate. As further shown, the scrubber 12 includes a solution outlet 22 and a gas outlet 24. Also, the system 10 includes an oxidation stage 26 for oxidizing pollutants in the flue gas to facilitate their removal from the flue gas. As shown, the oxidation stage 26 is interconnected between the power plant 18 and the scrubber 12.

As further shown, the system 10 includes a bioreactor 28 comprised of at least one chemostat 30 for growing algae cells (exemplary cells depicted at 32) and a plug flow reactor 34 for treating the algae cells 32 to trigger cell production of triglycerides. Preferably, and as shown, both the chemostat 30 and the plug flow reactor 34 are open raceways, though closed systems are also contemplated. Further, such open systems 10 can cover several acres of land to optimize economies of scale. For purposes of the present invention, the system 10 includes an algae separator 36 for removing the algae cells 32 from the plug flow reactor 34.

As shown in FIG. 1, the chemostat 30 includes a conduit 38. As further shown, the conduit 38 is provided with an input port 40 that is in fluid communication with the solution outlet 22 of the scrubber chamber 14. For purposes of the present invention, the input port 40 is also in communication with a reservoir (not illustrated) holding a nutrient mix (indicated by arrow 42). Preferably, the nutrient mix 42 includes phosphorous, nitrogen, sulfur and numerous trace elements necessary to support algae growth that are not provided to the bioreactor 28 by the scrubber solution 20. Further, the chemostat 30 is provided with a paddlewheel 44 for causing the medium 46 formed by the scrubber solution 20 and the nutrient mix 42 to continuously circulate around the conduit 38 at a predetermined fluid flow velocity. Also, each conduit 38 is provided with an output port 48 in communication with the plug flow reactor 34.

As shown, the plug flow reactor 34 includes an input port 50a for receiving overflow medium (indicated by arrow 46′) with algae cells 32 from the output port 48 of the chemostat 30. As further shown, the plug flow reactor 34 includes a conduit 52 for passing the medium 46″ with algae cells 32 downstream. The flow rate of the medium 46″ is due solely to gravity and the force of the incoming overflow medium 46′ from the chemostat 30. Preferably, the plug flow reactor 34 has a substantially fixed residence time of about one to four days. For purposes of the present invention, the system 10 is provided with a reservoir (not shown) that holds a modified nutrient mix (indicated by arrow 54). Further, the conduit 52 is provided with an input port 50b for receiving the modified nutrient mix 54. In order to manipulate the cellular behavior of algae cells 32 within the plug flow reactor 34, the modified nutrient mix 54 may contain a limited amount of a selected constituent, such as nitrogen or phosphorous. For instance, the nutrient mix 54 may contain no nitrogen. Alternatively, the algae cells 32 may exhaust nutrients such as nitrogen or phosphorous in the nutrient mix 42 at a predetermined point in the plug flow reactor 34. By allowing such nutrients to be exhausted, desired behavior in the algae cells 32 can be caused without adding a specific modified nutrient mix 54. Further, simply water can be added through the modified nutrient mix 54 to compensate for evaporation. In addition to input ports 50a and 50b, the conduit 52 is further provided with an input port 50c to receive other matter.

In FIG. 1, the algae separator 36 is shown in fluid communication with the conduit 52 of the plug flow reactor 34. For purposes of the present invention, the algae separator 36 separates the algae cells 32 from the medium 46″ and the remaining nutrients therein through flocculation and/or filtration. As further shown, the algae separator 36 includes an effluence outlet 56 and an algae cell outlet 60. For purposes of the present invention, the system 10 includes a channel 58 providing fluid communication between the effluence outlet 56 and the scrubber 12 through a solution input port 16b in the scrubber chamber 14.

Also, the system 10 includes a cell lysis apparatus 62 that receives algae cells 32 from the algae outlet 60 of the algae separator 36. As shown, the cell lysis apparatus 62 is in fluid communication with an oil separator 64. For purposes of the present invention, the oil separator 64 is provided with two outlets 66, 68. As shown, the outlet 66 is connected to a hydrolysis apparatus 70. Further, the hydrolysis apparatus 70 is connected to the input port 40 in the conduit 38 of the chemostat 30.

Referring back to the oil separator 64, it can be seen that the outlet 68 is connected to a biofuel reactor 72. It is further shown that the biofuel reactor 72 includes two exits 74, 76. For purposes of the present invention, the exit 74 is connected to the input port 50c in the conduit 52 of the plug flow reactor 34. Additionally or alternatively, the exit 74 may be connected to the input port 40 in the chemostat 30. Further, the exit 76 may be connected to a tank or reservoir (not shown) for purposes of the present invention.

In operation of the present invention, pollutant-contaminated flue gas (indicated by arrow 78) is directed from the power plant 18 to the oxidation stage 26. At the oxidation stage 26, nitrogen monoxide in the flue gas 78 is oxidized by nitric acid or by other catalytic or non-catalytic technologies to improve the efficiency of its subsequent removal. Specifically, nitrogen monoxide is oxidized to nitrogen dioxide. Thereafter, the oxidized flue gas (indicated by arrow 80) is delivered from the oxidation stage 26 to the scrubber 12. Specifically, the oxidized flue gas 80 enters the chamber 14 of the scrubber 12 through the input port 16a. Upon the entrance of the flue gas 80 into the chamber 14, the scrubber solution 20 is sprayed within the chamber 14 to adsorb or otherwise trap the pollutants in the flue gas 80 as is known in the field of scrubbing. With its pollutants removed, the clean flue gas (indicated by arrow 82) exits the scrubber 12 through the gas outlet 24. At the same time, the scrubber solution 20 and the pollutants exit the scrubber 12 through the solution outlet 22.

After exiting the scrubber 12, the scrubber solution 20 and pollutants (indicated by arrow 84) enter the chemostat 30 through the input port 40. Further, the nutrient mix 42 is fed to the chemostat 30 through the input port 40. In the conduit 38 of the chemostat 30, the nutrient mix 42, scrubber solution 20 and pollutants form the medium 46 for growing the algae cells 32. This medium 46 is circulated around the conduit 38 by the paddlewheel 44. Further, the conditions in the conduit 38 are maintained for maximum algal growth. For instance, in order to maintain the desired conditions, the medium 46 and the algae cells 32 are moved around the conduit 38 at a preferred fluid flow velocity of approximately fifty centimeters per second. Further, the amount of algae cells 32 in the conduit 38 is kept substantially constant. Specifically, the nutrient mix 42 and the scrubber solution 20 with pollutants are continuously fed at selected rates into the conduit 38 through the input port 40, and an overflow medium 46′ containing algae cells 32 is continuously removed through the output port 48 of the conduit 38.

After entering the input port 50a of the plug flow reactor 34, the medium 46″ containing algae cells 32 moves downstream through the conduit 52 in a plug flow regime. Further, as the medium 46″ moves downstream, the modified nutrient mix 54 may be added to the conduit 52 through the input port 50b. This modified nutrient mix 54 may contain a limited amount of a selected constituent, such as nitrogen or phosphorous. The absence or small amount of the selected constituent causes the algae cells 32 to focus on energy storage rather than growth. As a result, the algae cells 32 form triglycerides.

At the end of the conduit 52, the algae separator 36 removes the algae cells 32 from the remaining effluence (indicated by arrow 86). Thereafter, the effluence 86 is discharged from the algae separator 36 through the effluence outlet 56. In order to recycle the effluence 86, it is delivered through channel 58 to the input port 16b of the scrubber 12 for reuse as the scrubber solution 20. Further, the removed algae cells (indicated by arrow 88) are delivered to the cell lysis apparatus 62. Specifically, the removed algae cells 88 pass out of the algae cell outlet 60 to the cell lysis apparatus 62. For purposes of the present invention, the cell lysis apparatus 62 lyses the removed algae cells 88 to unbind the oil therein from the remaining cell matter. After the lysing process occurs, the unbound oil and remaining cell matter, collectively identified by arrow 90, are transmitted to the oil separator 64. Thereafter, the oil separator 64 withdraws the oil from the remaining cell matter as is known in the art. After this separation is performed, the oil separator 64 discharges the remaining cell matter (identified by arrow 92) out of the outlet 66 of the oil separator 64 to the input port 40 of the chemostat 30.

In the chemostat 30, the remaining cell matter 92 is utilized as a source of nutrients and energy for the growth of algae cells 32. Because small units of the remaining cell matter 92 are more easily absorbed or otherwise processed by the growing algae cells 32, the remaining cell matter 92 may first be broken down before being fed into the input port 40 of the chemostat 30. To this end, the hydrolysis apparatus 70 is interconnected between the oil separator 64 and the chemostat 30. Accordingly, the hydrolysis apparatus 70 receives the remaining cell matter 92 from the oil separator 64, hydrolyzes the received cell matter 92, and then passes hydrolyzed cell matter (identified by arrow 94) to the chemostat 30.

Referring back to the oil separator 64, it is recalled that the remaining cell matter 92 was discharged through the outlet 66. At the same time, the oil withdrawn by the oil separator 64 is discharged through the outlet 68. Specifically, the oil (identified by arrow 96) is delivered to the biofuel reactor 72. In the biofuel reactor 72, the oil 96 reacts with alcohol, such as methanol, to create mono-alkyl esters, i.e., biofuel fuel. This biofuel fuel (identified by arrow 98) is released from the exit 76 of the biofuel reactor 72 to a tank, reservoir, or pipeline (not shown) for use as fuel. In addition to the biofuel fuel 98, the reaction between the oil 96 and the alcohol produces glycerin as a byproduct. For purposes of the present invention, the glycerin (identified by arrow 100) is pumped out of the exit 74 of the biofuel reactor 72 to the input port 50c of the plug flow reactor 34.

In the plug flow reactor 34, the glycerin 100 is utilized as a source of carbon by the algae cells 32. Importantly, the glycerin 100 does not provide any nutrients that may be limited to induce oil production by the algae cells 32 or to trigger flocculation. The glycerin 100 may be added to the plug flow reactor 34 at night to aid in night-time oil production. Further, because glycerin 100 would otherwise provide bacteria and/or other non-photosynthetic organisms with an energy source, limiting the addition of glycerin 100 to the plug flow reactor 34 only at night allows the algae cells 32 to utilize the glycerin 100 without facilitating the growth of foreign organisms. As shown in FIG. 1, the exit 74 of the biofuel reactor 72 may also be in fluid communication with the input port 40 of the chemostat 30 (connection shown in phantom). This arrangement allows the glycerin 100 to be provided to the chemostat 30 as a carbon source.

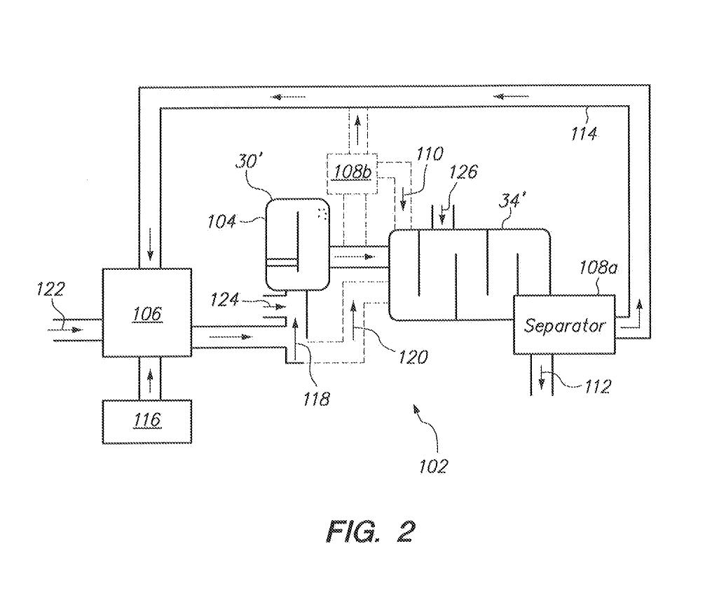

FIG. 2 shows that a closed-loop system 102 for growing algae can include a bioreactor 104 for growing algae cells in an aqueous solution having a chemostat 30′ (as described above) and a plug flow reactor 34′ (as described above). For example, the system 102 can produce algae, such as a micro-algae having a high oil content, for biofuel production. For the system 102, the aqueous solution used in either the chemostat 30′, the plug flow reactor 34′ or both, can be formulated such that the principal source of carbon for algae growth is supplied by sodium bicarbonate. For example, in some implementations, at least 50 percent of the carbon supplied for algae growth is supplied by sodium bicarbonate. Preferably, over 90 percent of the carbon supplied for algae growth is supplied by sodium bicarbonate. During algae growth in the aqueous solution, the concentration of sodium bicarbonate in the solution is reduced while the concentration of sodium carbonate increases.

FIG. 2 also shows that a regenerator 106 can be provided to regenerate sodium bicarbonate from the sodium carbonate. As shown, a separator 108a can be included in the system 102 for concentrating bioreactor algae. The separation can occur at the output of the plug flow reactor 34′, at the output of the chemostat 30′ (separator 108b), or at both locations. This results in concentrated algae and an algae depleted media. Algae concentrated by separator 108b can be input into the plug flow reactor 34′ (arrow 110) while algae concentrated by separator 108a can be forwarded (arrow 112) as an end product or to a biofuel reactor as shown in FIG. 1. Once separated from the algae, the algae depleted media is transported to the regenerator 106 via channel 114, as shown. At the regenerator 106, the algae depleted media is treated to regenerate sodium bicarbonate from the sodium carbonate. More specifically, carbon dioxide from a carbon dioxide source 116 can be introduced into the algae depleted media to regenerate sodium bicarbonate from the sodium carbonate according to the equation; Na₂CO₃+CO₂+H₂O→2 NaHCO₃. The regenerated sodium bicarbonate is then directed into the bioreactor 104 to supply carbon for further algae growth. The regenerated sodium bicarbonate can be introduced into the chemostat 30′ (arrow 118), the plug flow reactor 34′ (arrow 120) or both. The carbon dioxide source can include one or more of liquid carbon dioxide, carbon dioxide from an ethanol plant, carbon dioxide from a gasification source or carbon dioxide from a combustion source. As illustrated in FIG. 1, a scrubber 12 that is used to scrub a power plant effluent containing carbon dioxide can introduce carbon dioxide into the algae depleted media.

FIG. 2 also shows that in addition to carbon dioxide, heterotrophic sources of carbon such as algae debris, glycerin or cellulosic sugar can be introduced into the algae depleted media in the regenerator 106 (arrow 122). Arrow 124 illustrates that a suitable nutrient mix which includes phosphorous, nitrogen, potassium, sulfur and numerous trace elements necessary to support algae growth can be provided to the chemostat 30′. Arrow 126 illustrates that a suitable nutrient mix which, for example, limits one or more growth nutrients in order to trigger the production of oil in the form of triglycerides in the algae cells can be provided to the plug flow reactor 34′.

The above described processes may be performed as batch or continuous processes. Although FIG. 2 shows individual vessels for the bioreactor 104, separator 108a,b and regenerator 106, it is to be appreciated that one or more of these functions may be performed in a common vessel. For example, in a batch process, separation and regeneration could be performed in the chemostat vessel, etc.

While the particular Algae Biofuel Carbon Dioxide Distribution System as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A closed-loop system for growing algae which comprises:
a bioreactor for growing algae cells in an aqueous solution, the solution having an initial sodium bicarbonate concentration sufficient to supply at least 50 percent of carbon used for algae growth from the sodium bicarbonate, the sodium bicarbonate being converted to sodium carbonate in the bioreactor during algae growth;
a separator receiving algae and solution from the bioreactor and producing an algae depleted media; and
a regenerator for receiving the algae depleted media, for introducing carbon dioxide into the algae depleted media to regenerate sodium bicarbonate from the sodium carbonate and for delivering the regenerated sodium bicarbonate to the bioreactor for algae growth therein.

2. A closed loop system as recited in claim 1 wherein the regenerator comprises a scrubber and wherein carbon dioxide from a power plant effluent is introduced into the algae depleted media in the scrubber.

3. A closed loop system as recited in claim 1 wherein liquid carbon dioxide is introduced into the algae depleted media in the regenerator.

4. A closed loop system as recited in claim 1 wherein carbon dioxide from an ethanol plant is introduced into the algae depleted media in the regenerator.

5. A closed loop system as recited in claim 1 wherein carbon dioxide from a gasification source is introduced into the algae depleted media in the regenerator.

6. A closed loop system as recited in claim 1 wherein carbon dioxide from a combustion source is introduced into the algae depleted media in the regenerator.

7. A closed loop system as recited in claim 1 wherein algae debris is introduced into the algae depleted media in the regenerator.

8. A closed loop system as recited in claim 1 wherein glycerin is introduced into the algae depleted media in the regenerator.

9. A closed loop system as recited in claim 1 wherein cellulosic sugar is introduced into the algae depleted media in the regenerator.

10. A closed loop system as recited in claim 1 wherein the carbon dioxide introduced into the algae depleted media in the regenerator is devoid of atmospheric carbon dioxide.

11. A closed loop system as recited in claim 1 wherein the solution has an initial sodium bicarbonate concentration sufficient to supply at least 90 percent of carbon used for algae growth from the sodium bicarbonate.

12. A system for producing biofuel from algae which comprises:
a bioreactor for growing algae cells in an aqueous solution, the solution having an initial sodium bicarbonate concentration sufficient to supply at least 50 percent of carbon used for algae growth from the sodium bicarbonate, the sodium bicarbonate being converted to sodium carbonate in the bioreactor during algae growth;
a separator coupled with the bioreactor, the separator producing concentrated algae and an algae depleted media;
a regenerator for receiving the algae depleted media, for introducing carbon dioxide into the algae depleted media to regenerate sodium bicarbonate from the sodium carbonate and for delivering the regenerated sodium bicarbonate to the bioreactor for further algae growth therein; and
a device for processing the concentrated algae to form biofuel.

13. A system as recited in claim 12 wherein the regenerator comprises a scrubber and wherein carbon dioxide from a power plant effluent is introduced into the algae depleted media in the scrubber.

14. A system as recited in claim 12 wherein the carbon dioxide introduced into the algae depleted media in the regenerator is selected from the group consisting of liquid carbon dioxide, carbon dioxide from an ethanol plant, carbon dioxide from a gasification source and carbon dioxide from a combustion source.

15. A system as recited in claim 12 wherein a material selected from the group of materials consisting of algae debris, glycerin and cellulosic sugar is introduced into the algae depleted media in the regenerator.

16. A system as recited in claim 12 wherein the bioreactor comprises:
at least one chemostat formed with a conduit for growing algae therein, wherein the chemostat includes an input port for receiving sodium bicarbonate and for receiving a nutrient mix to form a medium for maximum algae growth, and wherein the chemostat has an output port for passing medium with algae growth from the conduit of the chemostat;
a means for continuously moving the medium through the conduit of the chemostat at a predetermined fluid flow velocity;
a plug flow reactor formed with a conduit having an input port for receiving the medium with algae growth from the chemostat; and
a means for adding a modified nutrient mix to the medium with algae growth in the plug flow reactor, wherein the modified nutrient mix comprises a limited amount of a selected constituent to trigger high oil production in the algae growth.

17. A system as recited in claim 16 wherein the device for processing the algae to form biofuel comprises:
an apparatus for lysing algae cells removed from the bioreactor to unbind oil within the algae cells;
an oil separator for withdrawing the oil from remaining cell matter; and
a reactor for receiving the oil from the oil separator and for synthesizing biofuel and glycerin from said oil.

18. A method for producing biofuel from algae, the method comprising the steps of:
growing algae in an aqueous solution in a bioreactor, the solution having a sodium bicarbonate concentration sufficient to supply at least 50 percent of carbon used for algae growth from the sodium bicarbonate, the sodium bicarbonate being converted to sodium carbonate in the bioreactor during algae growth;
concentrating algae to produce an algae depleted media;
introducing carbon dioxide into the algae depleted media to regenerate sodium bicarbonate from the sodium carbonate;
delivering regenerated sodium bicarbonate to the bioreactor for further algae growth therein; and
processing algae to form biofuel.

19. A method as recited in claim 18 wherein the introducing step is accomplished with a scrubber and wherein carbon dioxide from a power plant effluent is introduced into the algae depleted media in the scrubber.

20. A method as recited in claim 18 wherein the carbon dioxide introduced into the algae depleted media in the introducing step is selected from the group consisting of liquid carbon dioxide, carbon dioxide from an ethanol plant, carbon dioxide from a gasification source and carbon dioxide from a combustion source.
